(19) United States
(12) Patent Application Publication
(10) Pub. No.: US 2020/0029966 A1
(43) Pub. Date: Jan. 30, 2020

(54) Surgical Stapling Device With Variable Height Staple Pushers

(71) Applicant: Covidien LP, Mansfield, MA (US)

(72) Inventors: Hui Zhan, Shanghai (CN); Xiao Zhou, Shanghai (CN)

(21) Appl. No.: 16/491,198

(22) PCT Filed: Mar. 9, 2017
(86) PCT No.: PCT/CN2017/076153

§ 371 Date: Sep. 5, 2019

(51) Int. Cl. A61B 17/072

(52) U.S. Cl. CPC A61B 17/07207; A61B 2017/07235; A61B 2017/07271; A61B 2017/07257; A61B 2017/07278

(57) ABSTRACT

A surgical stapling device includes an anvil having a tissue engaging surface, a cartridge assembly including a staple cartridge, a firing mechanism for ejecting staples from the staple cartridge, and an actuator or lever that is configured to move the anvil and the cartridge assembly between spaced and clamped positions. The staple cartridge includes a plurality of rows of pushers that extend longitudinally within the staple cartridge and support the staples. In embodiments, the distance between the pushers and the tissue engaging surface of the anvil in at least one of the plurality of rows of pushers changes to compensate for an increase in outward deflection of the anvil in relation to the cartridge assembly along the longitudinal axis in the distal direction. In some embodiments, the distance between the pushers and the tissue engaging surface of the anvil in at least one of the plurality of rows of pushers is smaller at the distal portion of the staple cartridge than at the proximal portion.

### BACKGROUND

### 1. Technical Description

The present disclosure is directed to a surgical stapling device including a tool assembly having pushers for ejecting staples from a staple cartridge of the stapling device. More particularly, the present disclosure is directed to a surgical stapling device including a tool assembly having pushers for ejecting staples from the staple cartridge of the surgical stapling device that have a height that varies along the longitudinal axis of the surgical stapling device.

### 2. Background of Related Art

Surgical stapling devices for dispensing staples to suture tissue during surgical procedures are well known. Linear type surgical stapling devices typically include a tool assembly having jaws that are movable in relation to each other between spaced and clamped positions. The jaws have a proximal ends that are pivotally coupled to each other and distal ends that are spaced longitudinally from their proximal ends. The tool assembly includes a staple cartridge that houses a plurality of staples and a firing mechanism that translates from a proximal end of the tool assembly to a distal end of the tool assembly to sequentially eject staples from the staple cartridge. The jaws of the tool assembly are supported at their proximal ends in cantilevered fashion. During clamping and firing of the surgical stapling device, in response to the forces applied to the jaws of the tool assembly, the jaws tend to deflect outwardly from each other a progressively greater distance in a distal direction. This outward deflection of the jaws in relation to each other may have an adverse impact on staple formation within the jaws of the surgical stapling device during firing of the stapling device.

A continuing need exists in the art for a surgical stapling device including a tool assembly that provides improved staple formation along the entire length of the tool assembly.

### SUMMARY

In one aspect of the disclosure, a cartridge assembly comprising includes a body defining a longitudinal axis, a central knife slot extending along the longitudinal axis, and first and second rows of staple receiving slots positioned on each side of the central knife slot. The body has a distal portion and a proximal portion. The cartridge assembly includes a plurality of staples, each of which are supported within a respective one of the plurality of staple receiving slots to form first and second rows of staples. The cartridge assembly also includes a plurality of pushers. Each of the plurality of pushers has a height and is supported within a respective one of the plurality of staple receiving slots. The height of a pusher of the plurality of pushers in the distal portion of the body in the first row of staple receiving slots is greater than the height of a pusher of the plurality of pushers in the proximal portion of the body in the first row of staple receiving slots.

In another aspect of the disclosure, a surgical stapling device includes an anvil half-section and a cartridge receiving half-section. The anvil-half-section has a proximal portion and a distal portion. The distal portion supports an anvil and the proximal portion supports a hand grip. The cartridge receiving half-section also includes a distal portion and a proximal portion. A firing assembly is supported on the proximal portion of the cartridge receiving half-section and a cartridge assembly or SULU is supported on the distal portion of the cartridge receiving half-section. The cartridge assembly includes a body defining a longitudinal axis, a central knife slot extending along the longitudinal axis, and first and second rows of staple receiving slots positioned on each side of the central knife slot. The body has a distal portion and a proximal portion. The cartridge assembly includes a plurality of staples, each being supported within a respective one of the plurality of staple receiving slots to form first and second rows of staples. The cartridge assembly also includes a plurality of pushers, each of the plurality of pushers having a height and being supported within a respective one of the plurality of staple receiving slots. The height of the pushers of the plurality of pushers in the distal portion of the body in the first row of staple receiving slots is greater than the height of a pusher of the plurality of pushers in the proximal portion of the body in the first row of staple receiving slots.

In embodiments, the first row of staple receiving slots defines an outer row of staple receiving slots and the second row of staple receiving slots defines an inner row of staple receiving slots, wherein the inner row of staple receiving slots is positioned between the outer row of staple receiving slots and the central knife slot.

In some embodiments, the cartridge assembly includes a middle row of staple receiving slots positioned between the inner and outer rows of staple receiving slots.

In certain embodiments, the height of a pusher of the plurality of pushers in the distal portion of the body in each of the inner and outer rows of staple receiving slots is greater than the height of a pusher of the plurality of pushers in the proximal portion of the body in a corresponding row of the plurality of rows of staple receiving slots.

In embodiments, the height of the pushers in each of the rows of staple receiving slots gets progressively smaller from a proximal end of the body to a distal end of the body.

In some embodiments, the plurality of pushers in each of the rows of staple receiving slots include three groups of pushers including a proximal group of pushers, a middle group of pushers, and a distal group of pushers, wherein the proximal group of pushers has a height that is less than a height of the middle group of pushers and the height of the middle group of pushers is less than a height of the distal group of pushers.

In certain embodiments, each of the plurality of pushers includes an upper end defining a recess that is configured to receive a back span of a respective one of the plurality of staples.

### BRIEF DESCRIPTION OF THE DRAWINGS

Various embodiments of the presently disclosed surgical stapling device are described herein below with reference to the drawings, wherein:

FIG. 1 is a side perspective view of an exemplary embodiment of the presently disclosed surgical stapling device;

FIG. 1A is a side perspective, exploded view of the surgical stapling device shown in FIG. 1;

FIG. 2 is an exploded, side perspective view of a cartridge assembly of the surgical stapling device shown in FIG. 1;

FIG. 3 is enlarged view of the indicated area of detail shown in FIG. 2;

FIG. 4 is a side perspective, exploded view of staples and pushers of the cartridge assembly shown in FIG. 2;

FIG. 5 is a top view of the cartridge assembly shown in FIG. 2 illustrating the pushers with the remaining components of the staple cartridge shown in phantom;

FIG. 6A is a side cross-sectional view taken along section line 6-6 of FIG. 5;

FIG. 6B is a side cross-sectional view taken along section line 6-6 of FIG. 5 illustrating another embodiment of the pushers of the presently disclosed surgical stapling device shown in FIG. 1;

FIG. 6C is a side cross-sectional view taken along section line 6-6 of FIG. 5 illustrating yet another embodiment of the pushers of the presently disclosed surgical stapling device shown in FIG. 1;

FIG. 7 is an enlarged view of the indicated area of detail shown in FIG. 6A;

FIG. 8 is a side, partial cross-sectional view of the surgical stapling device shown in FIG. 1 with jaws of the surgical stapling device clamped about tissue;

FIG. 9 is a side, cross-sectional view taken through a distal portions of the anvil half-section and the cartridge receiving half-section with the surgical stapling device shown in FIG. 1 in the clamped positions;

FIG. 10 an enlarged view of the indicated area of detail shown in FIG. 9; and

FIG. 11 is an enlarged view of the indicated area of detail shown in FIG. 9.

### DETAILED DESCRIPTION OF EMBODIMENTS

The presently disclosed surgical stapling device will now be described in detail with reference to the drawings in which like reference numerals designate identical or corresponding elements in each of the several views. In this description, the term “proximal” is used generally to refer to that portion of the device that is closer to a clinician, while the term “distal” is used generally to refer to that portion of the device that is farther from the clinician. In addition, the term “endoscopic” is used generally to refer to endoscopic, laparoscopic, arthroscopic, and/or any other procedure conducted through small diameter incision or cannula. In addition, the term clinician is used generally to refer to medical personnel including doctors, nurses, and support personnel.

The presently disclosed surgical stapling device includes an anvil having a tissue engaging surface, a cartridge assembly including a staple cartridge, a firing mechanism for ejecting staples from the staple cartridge, and an actuator or lever that is configured to move the anvil and the cartridge assembly between a spaced position and a clamped position. The staple cartridge defines a longitudinal axis and includes a plurality of rows of staple receiving slots that are aligned with the longitudinal axis and receive rows of pushers and staples. The firing mechanism is configured to advance cam members through the staple cartridge into engagement with the rows of pushers to sequentially eject the rows of staples from the staple cartridge. In embodiments, the height of the pushers in at least one of the plurality of rows staple receiving slots increases in step fashion or progressively along the longitudinal axis of the staple cartridge in the distal direction to compensate for an increase in outward deflection of the anvil in relation to the cartridge assembly during clamping and firing of the surgical stapling device. In some embodiments, the height of the pushers in each of the plurality of rows of staple receiving slots of the staple cartridge increases in step fashion or progressively along the longitudinal axis of the staple cartridge from the proximal end to the distal end of the staple cartridge.

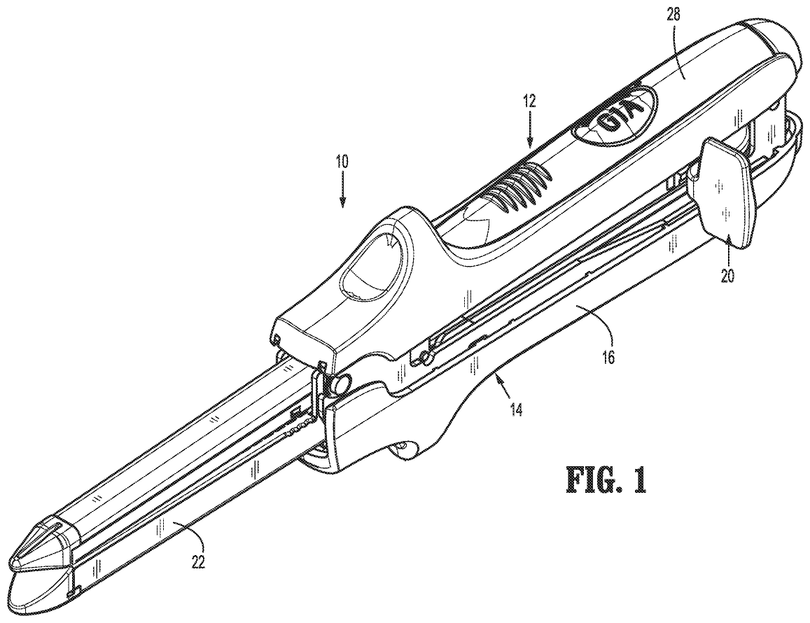

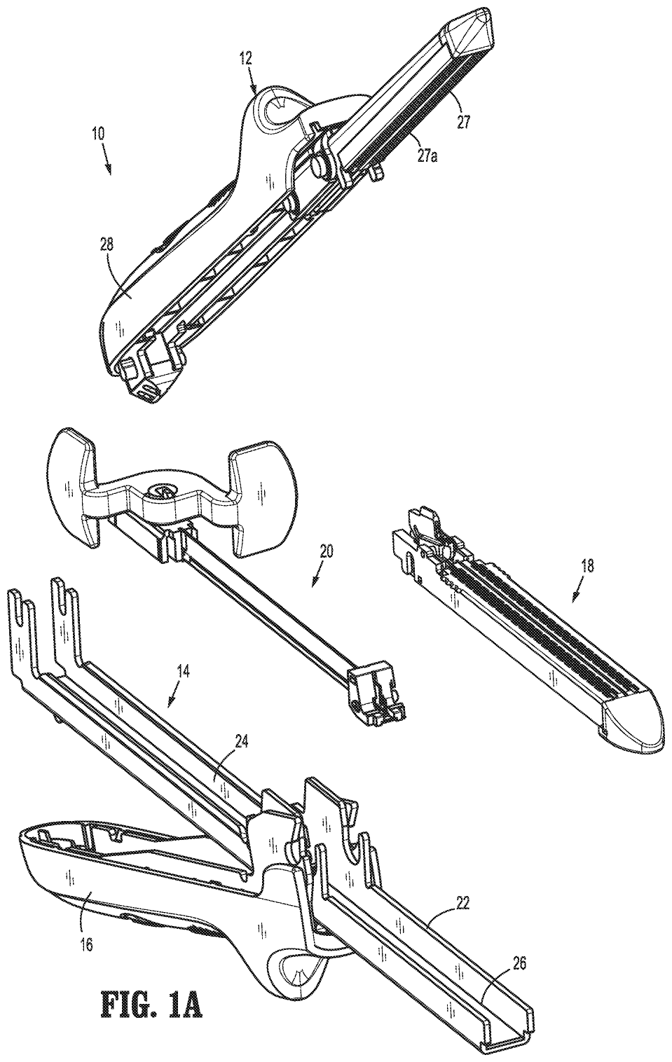

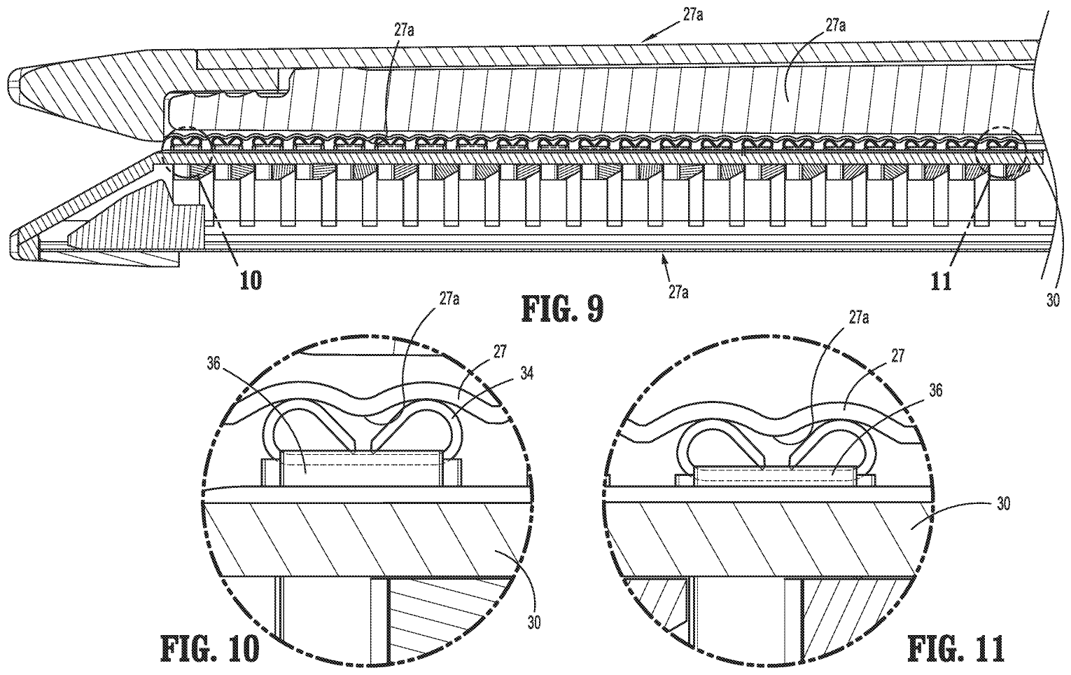

FIGS. 1-11 illustrate exemplary embodiments of the presently disclosed surgical stapling device designated generally as stapling device 10. Referring specifically to FIGS. 1 and 1A, the stapling device 10 includes an anvil half-section 12, a cartridge receiving half-section 14 supporting a clamping lever 16, a cartridge assembly or single use loading unit 18 (hereinafter “SULU”) and a firing assembly 20. The cartridge receiving half-section 14 includes a channel member 22 having a proximal portion 24 and a distal portion 26 (FIG. 1A). The proximal portion 24 of the channel member 22 receives the firing assembly 20 and pivotally supports the clamping lever 16. The distal portion 26 of the channel member 22 receives the SULU 18. The anvil half-section 12 includes a distal portion that supports an anvil 27 having a tissue contact surface 27a and a proximal portion that supports a hand grip 28. The tissue contact surface 27a of the anvil 27 defines a plurality of staple deforming recesses and is movable into opposition with the SULU 18 upon actuation of the clamp lever 16.

The clamp lever 16 is pivotally supported on the cartridge receiving half-section 14 and is engageable with the anvil half-section 12 such that movement of the clamping lever 16 from an unclamped position (FIG. 1A) to a clamped position (FIG. 1) moves the anvil half-section 12 into close approximation with the cartridge receiving half-section 14. Detailed operation and construction of surgical stapling devices of similar construction are described in U.S. Pat. No. 8,505,801 ('801 patent) and U.S. Pat. No. 7,721,933 ('933 patent) which are incorporated herein by reference in their entirety.

In embodiments, the anvil half-section 12 and the cartridge receiving half-section 14 are constructed to be reusable components and, as such, can be constructed from a biocompatible material suitable for sterilization and repeated use, e.g., stainless steel. In contrast, the SULU 18 and the firing assembly 20 can be constructed to be disposable and, as such, can be constructed from any suitable biocompatible material, e.g., plastics, metals, combinations thereof, having the requisite strength characteristics.

Although the presently disclosed surgical stapling device 10 is illustrated as an open-type linear surgical stapler, it is envisioned that the benefits of the present disclosure may be incorporated into a variety of different types of surgical stapling devices. The benefits of the presently disclosed stapling device are particularly relevant to stapling devices having linear or curved cantilevered jaws that do not include a dynamic clamping member or I-beam for controlling tissue gap.

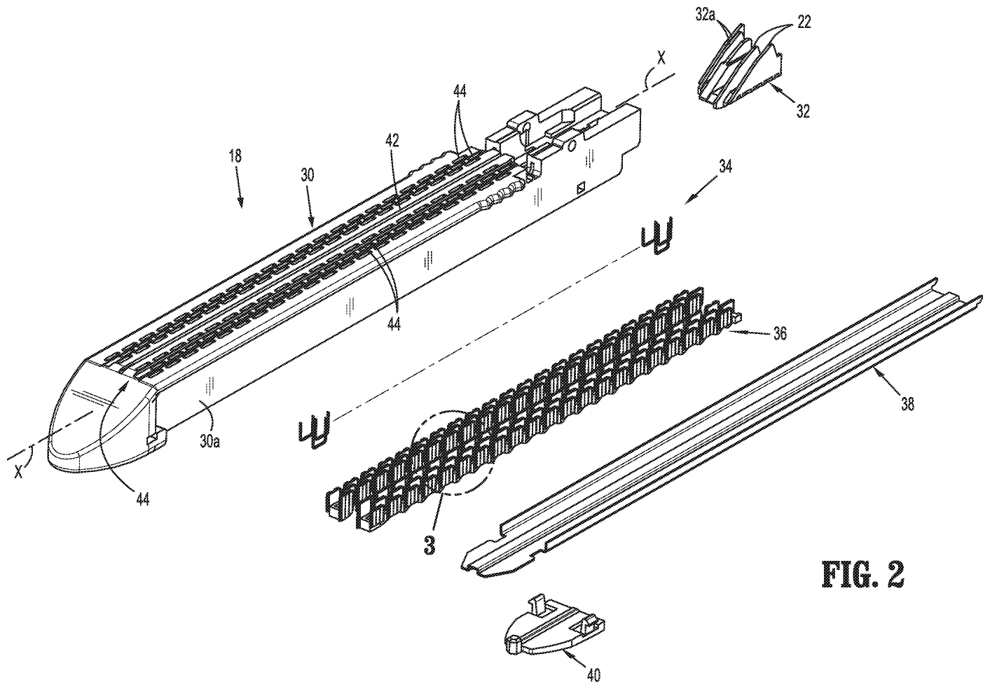

Referring to FIGS. 2-5, the SULU 18 (FIG. 2) includes a staple cartridge 30 having a body 30a defining a longitudinal axis “X”, an actuation sled 32, a plurality of rows of staples 34, a plurality of rows of pushers 36, a cover 38, and a securement member 40. The actuation sled 32 is configured to releasably engage the firing mechanism 20 when the SULU 18 22 is received within the distal portion 26 of the channel member 22 (FIG. 1A). It is envisioned that the actuation sled 32 may be replaced by one or more cam bars (not shown) as is known in the art.

The body 30a of the staple cartridge 30 defines a centrally located knife slot 42 (FIG. 2) and a plurality of rows of staple receiving slots 44 positioned on each side of the central knife slot 42. Each of the plurality of rows of staple receiving slots 44 extends in a direction substantially parallel to the longitudinal axis “X” of the staple cartridge 30. In some embodiments, the plurality of rows of staple receiving slots 44 includes three rows of staple receiving slots 44 on each side of the central knife slot 42 including an inner row 44a of staple receiving slots 44 located closest to the central knife slot 42, an outer row 44c of staple receiving slots 44 located furthest from the central knife slot 42, and a middle row 44b of staple receiving slots 44 located between the inner and outer rows 44a, 44c of staple receiving slots 44. Alternately, the body 30a of the staple cartridge 30 can define any number of rows of staple receiving slots 44 on each side of the central knife slot 42, e.g., 1-6 rows.

As is known in the art, the actuation sled 32 (FIG. 2) includes a plurality of spaced cam members 32a-b positioned on each side of the central knife slot 42. The cam members 32a-b are positioned to translate through channels (not shown) in the body 30a of the staple cartridge 30 into engagement with the pushers 36 to lift the pushers 36 within the staple receiving slots 44 and eject the staples 34 from the staple cartridge 30.

Each of the staple receiving slots 44 (FIG. 2) receives a staple 34 and a pusher 36. Each of the staples 34 includes a back span 50 and a leg 52 (FIG. 4) extending from each end of the back span 50. In embodiments, an upper end of each of the pushers 36 defines a recess 54. When the staples 34 and the pushers 36 are received within the staple receiving slots 44 of the staple cartridge 30, the back span 50 of each staple 34 is supported in a recess 54 of a respective one of the plurality of pushers 36.

In embodiments, the pushers 36 may be secured to adjacent pushers 36 (FIG. 4) such that a plurality of pushers 36 are secured together. In the embodiment shown, three pushers 36 are secured together via cam members 56 to define a triple pusher 60. The cam members 56 (FIG. 4) are provided with cam surfaces 56a that are positioned to be engaged by the cam members 32a-b of the actuation sled 32 (FIG. 2) to eject the staples 34 sequentially from the staple cartridge 30 through the staple receiving slots 44. In embodiments, the triple pusher 60 a pusher 36 that is received in each of the inner row 44a of staple receiving slots 44, the middle row 44b of staple receiving slots 44, and the outer row 44c of staple receiving slots 44. Alternately, the pushers 36 can be individually supported within the staple cartridge 30 or secured together and supported in groups of two or more pushers 36.

In embodiments, the plurality of rows of staples 34 includes staples 34a that are received in the inner row 44a of staple receiving slots 44, staples 34b that are received in the middle row 44b of staple receiving slots 44, and staples 34c that are received in the outer row 44c of the staple receiving slots 44 of the staple cartridge 30. The staples 34a-c can be different sizes to vary the amount of compression applied to tissue in the inner, middle, and outer rows of staples 44a-c. Similarly, the pusher heights in each of the rows 44a-44c of the staple receiving slots 44 of the staple cartridge 30 may also vary in height. In addition, although not shown, the tissue contacting surface 27a of the anvil 27 may be linear or stepped. The heights of the staples 34 and the pushers 38, and the configuration of the tissue contact surface of the anvil 27 can be selected to control the compression of tissue treated with the surgical stapling device 10 along each of the rows 44a-c of the staple receiving slots 44. U.S. Pat. No. 7,828,189 ('189 patent) discloses different combinations of staple and pusher sizes and different anvil contacting surface tissue configurations to achieve a desired tissue compression along the different rows of each of the staple receiving slots. The '189 patent is incorporated herein by reference in its entirety,

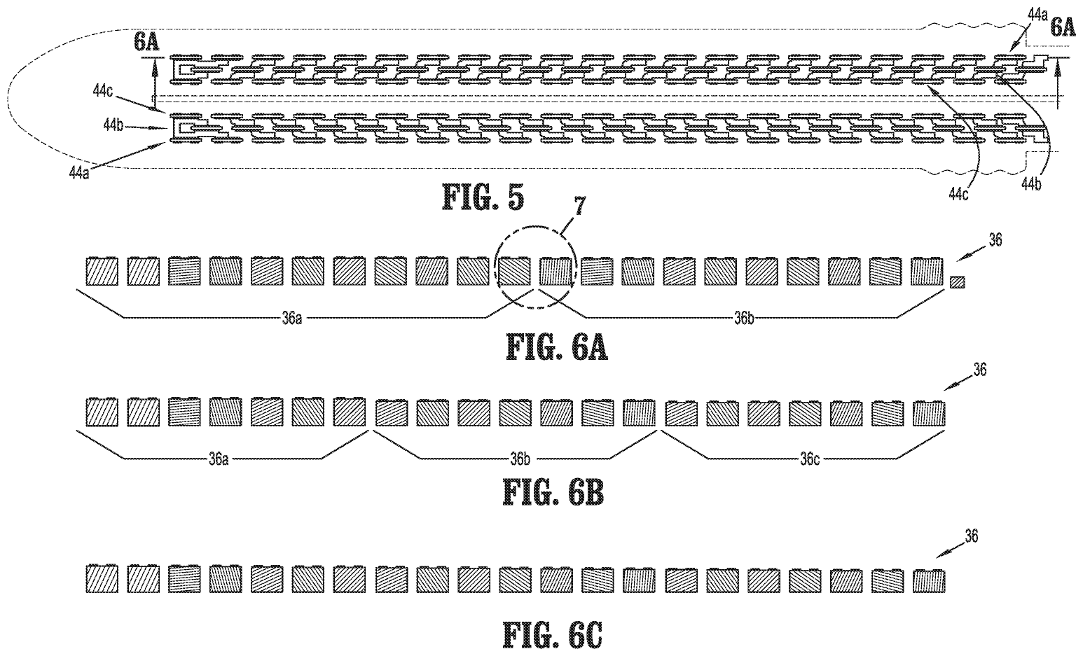

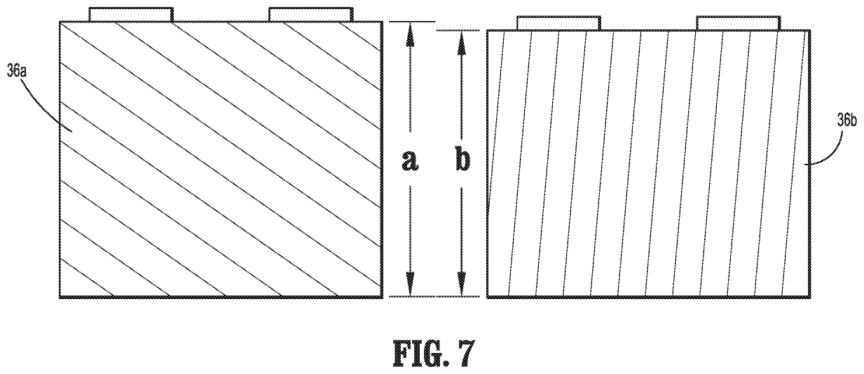

Referring to FIGS. 5-7, the pushers 36 located in a proximal portion of each row of the staple receiving slots 44 have a height “b” (FIG. 7) that is smaller than the height “a” (FIG. 7) of the pushers 36 located in a distal portion of each row of the staple receiving slots 44. By providing pushers 36 with a larger height in the distal portion of each row of the staple receiving slots 44 of the staple cartridge 30 than the height of the pushers 36 in the proximal portion of each row of staple receiving slots 44, outward deflection of the anvil 27 in relation to the staple cartridge 30 at the distal portion of the surgical stapling device 10 can be compensated for to achieve a desired degree of tissue compression along each rows of staples 34. Although the row of pushers 36 shown in FIG. 6 is shown to have two groups of pushers 36a and 36b having different pusher heights “a” and “b”, where “a” is greater than “b”, it is envisioned that the pushers 36a-c may be divided into multiple groups of pushers of different heights, e.g., 3 groups 36a-c (FIG. 6B) or more, with the heights of the pushers 36 of the pusher groups increasing in the distal direction along the staple cartridge 30 (FIG. 6B). It is also envisioned that the pushers 36 can have a height that progressively increases from a proximal end of the staple cartridge 30 to a distal end of the staple cartridge 30 in any one or all of the rows of staple receiving slots 44 of the staple cartridge 30 (FIG. 6C).

It is envisioned that each of the rows 44a-c (FIG. 5) of staple receiving slots 44 need not have pushers that vary in height along the longitudinal axis of the staple cartridge 30. For example, the heights of the pushers 36 may vary along the longitudinal axis of the staple cartridge 30 in any one or all of the rows 44a-c of staple receiving slots 44.

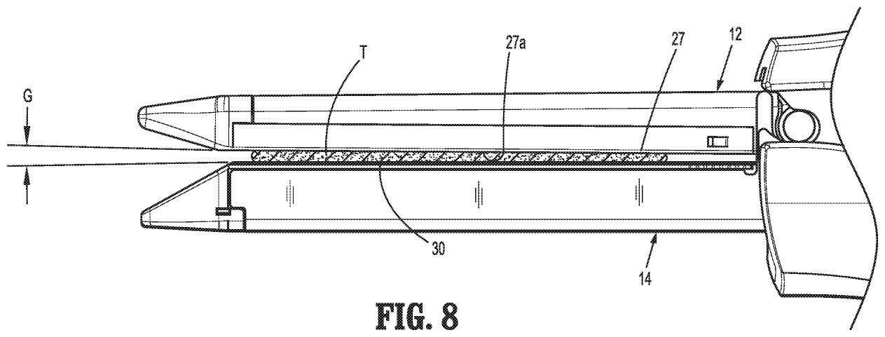

Referring to FIGS. 8-11, when tissue “T” is clamped between the anvil 27 of the anvil half-section 12 and the staple cartridge 30 of the cartridge receiving half-section 14, a gap “G” (FIG. 8) is defined between the tissue contact surface of the anvil 27 and the staple cartridge 30. This gap “G” progressively increases in height from a proximal end of the staple cartridge 30 towards a distal end of the staple cartridge 30. The increase in the height of the gap “G” results from outward deflection of a distal portion of the cartridge receiving half-section 14 in relation to a distal portion of the anvil half-section 12. More specifically, because the distal portions of the anvil half-section 12 and cartridge receiving half-section 14 are supported in cantilevered fashion, the distal portions of the anvil half-section 12 and cartridge receiving half-section 14 deflect outwardly in the distal direction as forces are applied in a direction transverse to the longitudinal axis “X” (FIG. 2) of the staple cartridge 30 to the anvil half-section 12 and cartridge receiving half-section 14.

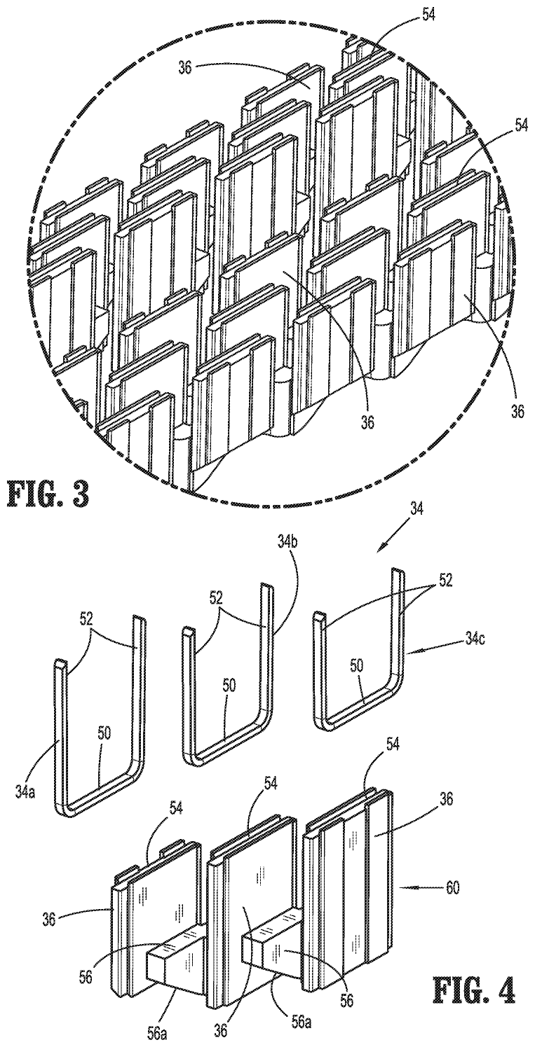

When tissue “T” is clamped between the anvil 27 of the anvil half-section 12 and the staple cartridge 30 of the cartridge receiving half-section 14 and the surgical stapling device 10 is fired to advance the actuation sled 32 through the staple cartridge 30, via actuation of the firing assembly 20 (FIG. 1A), the cam members 32a-b of the cam sled 32 (FIG. 3) translate through the channels (not shown) defined in the staple cartridge 30 into engagement with the cam members 56 (FIG. 4) of the pushers 36 to lift the pushers 36 within the staple receiving slots 44 of the staple cartridge 30 and eject the staples 34 from the staple cartridge 30. During clamping of tissue “T”, the tissue “T” exerts a force on the staple cartridge 30 and the anvil 27 to cause outward deflection of the staple cartridge 30 and the anvil 27 in relation to each other. This results in a tissue gap “G” (FIG. 8) between the staple cartridge 30 and the anvil 27 that increases in a distal direction of the staple cartridge 30. By increasing the height of the pushers 36 in the distal portion of each of the rows of staple receiving slots 44 of the staple cartridge 30, the increase in gap “G” in the distal portion of the staple cartridge 30 can be compensated for to provide a similar formation of staples in tissue clamped in the distal portion of the gap “G” to that of tissue clamped in the proximal portion of the tissue gap “G”.

Persons skilled in the art will understand that the devices and methods specifically described herein and illustrated in the accompanying drawings are non-limiting exemplary embodiments. It is envisioned that the elements and features illustrated or described in connection with one exemplary embodiment may be combined with the elements and features of another without departing from the scope of the present disclosure. As well, one skilled in the art will appreciate further features and advantages of the disclosure based on the above-described embodiments. Accordingly, the disclosure is not to be limited by what has been particularly shown and described, except as indicated by the appended claims.

What is claimed is:

1. A cartridge assembly comprising;
a body defining a longitudinal axis, a central knife slot extending along the longitudinal axis, and first and second rows of staple receiving slots positioned on each side of the central knife slot, the body having a distal portion and a proximal portion;
a plurality of staples, each of the plurality of staples being supported within a respective one of the plurality of staple receiving slots to form first and second rows of staples; and
a plurality of pushers, each of the plurality of pushers having a height and being supported within a respective one of the plurality of staple receiving slots, wherein the height of a pusher of the plurality of pushers in the distal portion of the body in the first row of staple receiving slots is greater than the height of a pusher of the of the plurality of pushers in the proximal portion of the body in the first row of staple receiving slots.

2. The cartridge assembly of claim 1, wherein the first row of staple receiving slots defines an outer row of staple receiving slots and the second row of staple receiving slots defines an inner row of staple receiving slots, the inner row of staple receiving slots being positioned between the outer row of staple receiving slots and the central knife slot.

3. The cartridge assembly of claim 2, further including a middle row of staple receiving slots positioned between the inner and outer rows of staple receiving slots.

4. The cartridge assembly of claim 1, wherein the height of a pusher of the plurality of pushers in the distal portion of the body in each of the inner and outer rows of staple receiving slots is greater than the height of a pusher of the plurality of pushers in the proximal portion of the body in a corresponding row of the plurality of rows of staple receiving slots.

5. The cartridge assembly of claim 1, wherein the height of the pushers in each of the inner and outer rows of staple receiving slots progressively gets smaller from a proximal end of the body to a distal end of the body.

6. The cartridge assembly of claim 1, wherein the plurality of pushers in each of the inner and outer rows of staple receiving slots include three groups of pushers including a proximal group of pushers, a middle group of pushers, and a distal group of pushers, wherein the proximal group of pushers has a height that is less than a height of the middle group of pushers and the height of the middle group of pushers is less than a height of the distal group of pushers.

7. The cartridge assembly of claim 1, wherein each of the plurality of pushers includes an upper end defining a recess, the recess being configured to receive a backspan of a respective one of the plurality of staples.

8. A surgical stapling device comprising:
an anvil half-section having a proximal portion and a distal portion, the distal portion supporting an anvil and the proximal portion supporting a hand grip;
a cartridge receiving half-section including a distal portion and a proximal portion;
a firing assembly supported on the proximal portion of the cartridge receiving half-section;
a cartridge assembly supported on the distal portion of the cartridge receiving half-section, the cartridge assembly including
a body defining a longitudinal axis, a central knife slot extending along the longitudinal axis, and first and second rows of staple receiving slots positioned on each side of the central knife slot, the body having a distal portion and a proximal portion;
a plurality of staples, each of the plurality of staples being supported within a respective one of the plurality of staple receiving slots to form first and second rows of staples; and
a plurality of pushers, each of the plurality of pushers having a height and being supported within a respective one of the plurality of staple receiving slots, wherein the height of a pusher of the plurality of pushers in the distal portion of the body in the first row of staple receiving slots is greater than the height of a pusher of the plurality of pushers in the proximal portion of the body in the first row of staple receiving slots.

9. The surgical stapling device of claim 8, wherein the first row of staple receiving slots defines an outer row of staple receiving slots and the second row of staple receiving slots defines an inner row of staple receiving slots, the inner row of staple receiving slots being positioned between the outer row of staple receiving slots and the central knife slot.

10. The surgical stapling device of claim 8, further including a middle row of staple receiving slots positioned between the inner and outer rows of staple receiving slots.

11. The surgical stapling device of claim 8, wherein the height of a pusher of the plurality of pushers in the distal portion of the body in each of the inner and outer rows of staple receiving slots is greater than the height of a pusher of the of the plurality of pushers in the proximal portion of the body in a corresponding row of the plurality of rows of staple receiving slots in the distal portion of the body.

12. The surgical stapling device of claim 8, wherein the height of the pushers in each of the inner and outer rows of staple receiving slots progressively gets smaller from a proximal end of the body to a distal end of the body.

13. The surgical stapling device of claim 8, wherein the plurality of pushers in each of the inner and outer rows of staple receiving slots include three groups of pushers including a proximal group of pushers, a middle group of pushers, and a distal group of pushers, wherein the proximal group of pushers has a height that is less than a height of the middle group of pushers and the height of the middle group of pushers is less than a height of the distal group of pushers.

14. The surgical stapling device of claim 8, wherein each of the plurality of pushers includes an upper end defining a recess, the recess being configured to receive a backspan of a respective one of the plurality of staples.

15. The surgical stapling device of claim 8, wherein the cartridge assembly includes an actuation sled.
